(19) United States
(12) Patent Application Publication
(10) Pub. No.: US 2025/0357003 A1
(43) Pub. Date: Nov. 20, 2025

(54) METHOD AND COMPUTER SYSTEM FOR ANALYZING GASTRIC ENDOSCOPIC IMAGE

(71) Applicants: NATIONAL CHENG KUNG UNIVERSITY, Tainan City (TW); TAINAN HOSPITAL, MINISTRY OF HEALTH AND WELFARE, Tainan City (TW)

(72) Inventors: Hsiu-Chi CHENG, Tainan City (TW); Chun-Rong HUANG, Tainan City (TW); Yu-Ching TSAI, Tainan City (TW); Hsiao-Bai YANG, Hsinchu County (TW); Jyun-Yao JHANG, Changhua County (TW); Tzu-Chun HSU, Taichung City (TW); Po-Hsiang HSU, Tainan City (TW)

(21) Appl. No.: 19/007,579

(22) Filed: Jan. 2, 2025

(30) Foreign Application Priority Data
May 15, 2024 (TW) 113117946

(51) Int. Cl. G16H 50/20; G06T 7/00

(52) U.S. Cl. CPC G16H 50/20; G06T 7/0012; G06T 2207/20084; G06T 2207/30092

(57) ABSTRACT

The present disclosure relates to a method for analyzing a gastric endoscopic image. The method includes: acquiring a gastric antrum endoscopic image, a gastric body endoscopic image, and a gastric cardia endoscopic image; inputting these images into a scaling feature fusion module which contains multiple shared-weights scaling sub-networks, each having a different field of view and outputting scale features of the antrum, body, and cardia, respectively; concatenating the scale features of the same section to obtain cross-view features of the antrum, body, and cardia; and inputting these cross-view features into a section correlation module to concatenate at least two features and generate a corpus-predominant gastritis index through a classifier.

### RELATED APPLICATIONS

This application claims priority to Taiwan Application Serial Number 113117946, filed on May 15, 2024, which is herein incorporated by reference.

### BACKGROUND

### Technical Field

The present disclosure relates to a method for analyzing a gastric endoscopic image, which may be utilized to calculate a corpus-predominant gastritis index.

### Description of Related Art

Gastric cancer is the sixth most common cancer in the world and the fourth leading cause of cancer death. Gastric cancer occurs after Helicobacter pylori (H. pylori) infection through inflammation of the gastric body, gastric atrophy, and gastric intestinal metaplasia. Since early diagnosis may improve disease survival rates, to regular follow-up of patients who have already suffered from significant gastric corpus inflammation after H. pylori infection is particularly important. How to correctly diagnose and grade gastric corpus inflammation, which is indicated by the corpus-predominant gastritis index, during gastroscopy is an important clinical issue. Currently, diagnosis of the corpus-predominant gastritis index requires biopsies from five to six locations in the stomach during gastroscopy, which is then interpreted by a pathologist. The advantage is that it may be correctly diagnosed through pathological interpretation, but the disadvantage is time-consuming, creating risks such as bleeding, and not suitable for large-scale screening. Therefore, developing a technology for quickly analyzing the corpus-predominant gastritis index without invasive biopsy is an important issue in precision health that still needs to be developed.

### SUMMARY

One aspect of the present disclosure relates to a method for analyzing a gastric endoscopic image, the method includes acquiring a gastric antrum endoscopic image, a gastric body endoscopic image, and a gastric cardia endoscopic image; inputting the gastric antrum endoscopic image, the gastric body endoscopic image, and the gastric cardia endoscopic image into a scaling feature fusion module which contains a plurality of shared-weights scaling sub-networks, each of the shared-weights scaling sub-networks having a different field of view, and utilized to receive the gastric antrum endoscopic image, the gastric body endoscopic image, and the gastric cardia endoscopic image, and output a gastric antrum scaling feature, gastric body scaling feature, and a gastric cardia scaling feature, respectively; concatenating the gastric antrum scaling feature output by the shared-weights scaling sub-networks to obtain a cross-view gastric antrum feature, and concatenating the gastric body scaling feature output by the shared-weights scaling sub-networks to obtain a cross-view gastric body feature, and concatenating the gastric cardia scaling feature output by the shared-weights scaling sub-networks to obtain a cross-view gastric cardia feature; and inputting the cross-view gastric antrum feature, the cross-view gastric body feature, and the cross-view gastric cardia feature into a section correlation module, wherein the section correlation module is utilized to concatenate at least two of the cross-view gastric antrum feature, the cross-view gastric body feature, and the cross-view gastric cardia feature, and the section correlation module includes a classifier to generate a corpus-predominant gastritis index (CGI).

In accordance with one or more embodiments of the present disclosure, one of the shared-weight scaling sub-networks includes a plurality of neural networks, the neural networks share weights and are utilized to receive the gastric antrum endoscopic image, the gastric body endoscopic image, and the gastric cardia endoscopic image, respectively.

In accordance with one or more embodiments of the present disclosure, one of the neural networks includes a convolutional layer, a residual block, a channel attention layer, or a pooling block.

In accordance with one or more embodiments of the present disclosure, the section correlation module is utilized to concatenate the cross-view gastric antrum feature and the cross-view gastric body feature to generate a first fusion feature, wherein the section correlation module is utilized to concatenate the cross-view gastric antrum feature, the cross-view gastric body feature, and the cross-view gastric cardia feature to generate a second fusion feature, and wherein the section correlation module is utilized to concatenate the cross-view gastric antrum feature and the cross-view gastric cardia feature to generate a third fusion feature.

In accordance with one or more embodiments of the present disclosure, the first fusion feature is input to the classifier to calculate a first loss, and wherein the second fusion feature is input to the classifier to calculate a second loss, and wherein the third fusion feature is input to the classifier to calculate a third loss.

In accordance with one or more embodiments of the present disclosure, the section correlation module is utilized to concatenate the first fusion feature, the second fusion feature, and the third fusion feature to obtain a sum feature, wherein the classifier is utilized to generate the corpus-predominant gastritis index based on the sum feature.

In accordance with one or more embodiments of the present disclosure, the sum feature is input to the classifier to calculate a fourth loss.

In accordance with one or more embodiments of the present disclosure, the first loss, the second loss, the third loss and the fourth loss are summed into a sum loss.

Another aspect of the present disclosure relates to a computer system, which includes a memory and a processor. The memory is configured to store a plurality of instructions. The processor is coupled to the memory, and configured to execute the instructions to perform the following steps: acquiring a gastric antrum endoscopic image, a gastric body endoscopic image, and a gastric cardia endoscopic image; inputting the gastric antrum endoscopic image, the gastric body endoscopic image, and the gastric cardia endoscopic image into a scaling feature fusion module which contains a plurality of shared-weights scaling sub-networks, each of the shared-weights scaling sub-networks having a different field of view, and utilized to receive the gastric antrum endoscopic image, the gastric body endoscopic image, and the gastric cardia endoscopic image, and output a gastric antrum scaling feature, gastric body scaling feature, and a gastric cardia scaling feature, respectively; concatenating the gastric antrum scaling feature output by the shared-weights scaling sub-networks to obtain a cross-view gastric antrum feature, and concatenating the gastric body scaling feature output by the shared-weights scaling sub-networks to obtain a cross-view gastric body feature, and concatenating the gastric cardia scaling feature output by the shared-weights scaling sub-networks to obtain a cross-view gastric cardia feature; and inputting the cross-view gastric antrum feature, the cross-view gastric body feature, and the cross-view gastric cardia feature into a section correlation module, wherein the section correlation module is utilized to concatenate at least two of the cross-view gastric antrum feature, the cross-view gastric body feature, and the cross-view gastric cardia feature, and the section correlation module includes a classifier to generate a corpus-predominant gastritis index (CGI).

In accordance with one or more embodiments of the present disclosure, one of the shared-weight scaling sub-networks includes a plurality of neural networks, the neural networks share weights and are utilized to receive the gastric antrum endoscopic image, the gastric body endoscopic image, and the gastric cardia endoscopic image, respectively.

In accordance with one or more embodiments of the present disclosure, one of the neural networks includes a convolutional layer, a residual block, a channel attention layer, or a pooling block.

In accordance with one or more embodiments of the present disclosure, the section correlation module is utilized to concatenate the cross-view gastric antrum feature and the cross-view gastric body feature to generate a first fusion feature, wherein the section correlation module is utilized to concatenate the cross-view gastric antrum feature, the cross-view gastric body feature, and the cross-view gastric cardia feature to generate a second fusion feature, and wherein the section correlation module is utilized to concatenate the cross-view gastric antrum feature and the cross-view gastric cardia feature to generate a third fusion feature.

In accordance with one or more embodiments of the present disclosure, the first fusion feature is input to the classifier to calculate a first loss, and wherein the second fusion feature is input to the classifier to calculate a second loss, and wherein the third fusion feature is input to the classifier to calculate a third loss.

In accordance with one or more embodiments of the present disclosure, the section correlation module is utilized to concatenate the first fusion feature, the second fusion feature, and the third fusion feature to obtain a sum feature, wherein the classifier is utilized to generate the corpus-predominant gastritis index based on the sum feature.

In accordance with one or more embodiments of the present disclosure, the sum feature is input to the classifier to calculate a fourth loss.

In accordance with one or more embodiments of the present disclosure, the first loss, the second loss, the third loss and the fourth loss are summed into a sum loss.

### BRIEF DESCRIPTION OF THE DRAWINGS

This disclosure can be more fully understood by reading the following detailed descriptions of the embodiments, with reference made to the accompanying drawings as follows:

FIG. 1 is a schematic diagram of a computer system in accordance with some embodiments of the present disclosure.

FIG. 2 is a schematic diagram illustrating the architecture of a scaling feature fusion module and a section correlation module in accordance with some embodiments of the present disclosure.

FIG. 3 is a schematic diagram of detection results in accordance with some embodiments of the present disclosure.

FIG. 4 is a flowchart of a method for analyzing a gastric endoscopic image in accordance with some embodiments of the present disclosure.

### DETAILED DESCRIPTION

Reference will now be made in detail to the present embodiments of this disclosure, examples of which are illustrated in the accompanying drawings. Wherever possible, the same reference numbers are utilized in the drawings and the description to refer to the same or like parts.

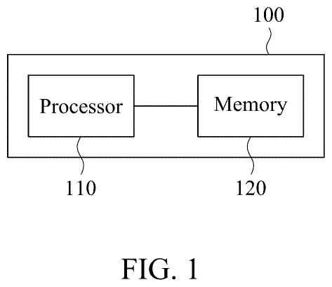

FIG. 1 is a schematic diagram of a computer system in accordance with some embodiments of the present disclosure. Referring to FIG. 1, the computer system 100 may be a tablet computer, a personal computer, a notebook computer, a server, a distributed computer, a cloud server, an industrial computer, a medical device, or various electronic devices with computing capabilities, but the present disclosure is not limited thereto. The computer system 100 includes a processor 110 and a memory 120 that is communicatively connected to the processor 110. The communication connection may be implemented by any wired or wireless communication means, or through the Internet. The processor 110 may be a central processing unit (CPU), a microprocessor, a microcontroller, a digital signal processor, an image processing chip, or a special application integrated circuit. The memory 120 may be a random access memory, a read-only memory, a flash memory, a floppy disk, a hard disk, an optical disk, a pen drive, a magnetic tape or a database accessible through the Internet, which stores multiple instructions, and the processor 110 may execute these instructions to complete the method for analyzing a gastric endoscopic image. The method for analyzing a gastric endoscopic image is illustrated below.

The method for analyzing a gastric endoscopic image in this disclosure may process gastric antrum endoscopic images, gastric body endoscopic images, and gastric cardia endoscopic images, and may also process different fields of view, and a more accurate corpus-predominant gastritis index may be calculated based on different sections and the different fields of view.

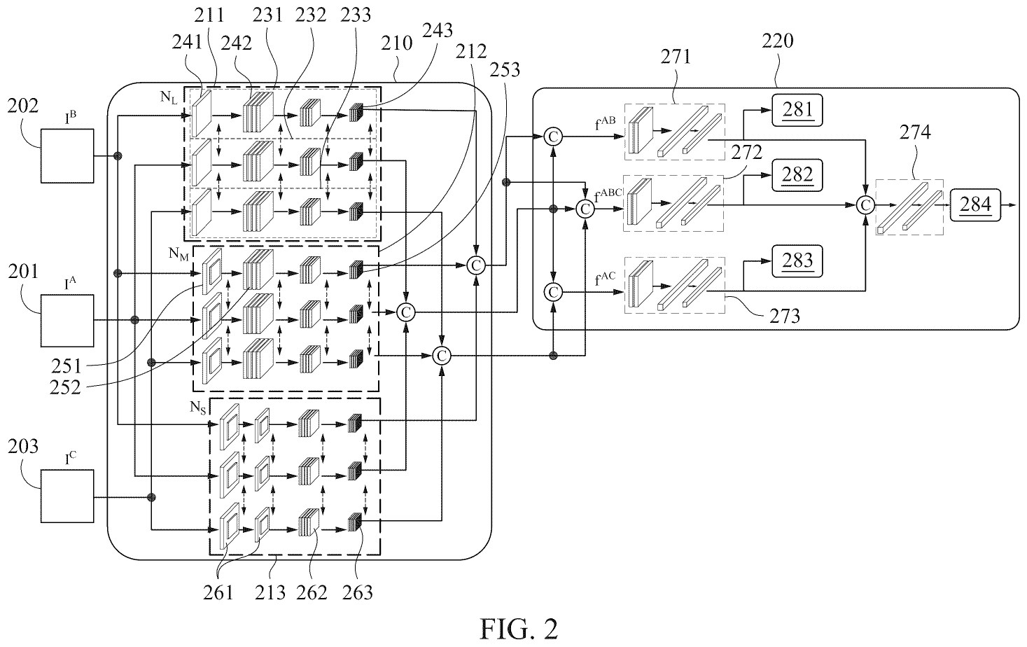

FIG. 2 is a schematic diagram illustrating the architecture of a scaling feature fusion module and a section correlation module in accordance with some embodiments of the present disclosure. Referring to FIG. 2, the machine learning model proposed in this disclosure is also referred to as a gastric section correlation network (GSCNet), and includes a scaling feature fusion module 210 and a section correlation module 220. First, the gastric antrum endoscopic image 201 (also expressed as image I^{A}), the gastric body endoscopic image 202 (also expressed as image I^{B}) and the gastric cardia endoscopic image 203 (also expressed as image I^{C}) are acquired, and these images are input to the scaling feature fusion module 210.

The scaling feature fusion module 210 includes shared-weights scaling sub-networks 211-213, and the fields of view of the shared-weights scaling sub-networks 211-213 are different from each other. Each of the shared-weights scaling sub-networks 211-213 receives the gastric antrum endoscopic image 201, the gastric body endoscopic image 202 and the gastric cardia endoscopic image 203. In this embodiment, three shared-weights scaling sub-networks are utilized. However, more or fewer shared-weights scaling sub-networks may be designed in other embodiments, and the present disclosure is not limited thereto.

Each of the shared-weights scaling sub-networks 211-213 includes neural networks. The neural networks in the same shared-weights scaling sub-network have the same architecture and share weights. These neural networks are utilized to receive the gastric antrum endoscopic image 201, the gastric body endoscopic image 202 and the gastric cardia endoscopic image 203, respectively. In other words, the number of the neural networks is the same as the number of the gastric endoscopic images. For example, the shared-weights scaling sub-network 211 includes neural networks 231-233. The neural network 231 receives the gastric body endoscopic image 202, the neural network 232 receives the gastric antrum endoscopic image 201, and the neural network 233 receives the gastric cardia endoscopic image 203. These three neural networks 231-233 share the weights, and the purpose thereof is to analyze the gastric antrum, the gastric body, and the gastric cardia at the same time to obtain a more comprehensive and general model and avoid the problem of over-fitting.

The architectures of neural networks in different shared-weight scaling sub-networks 211-213 are different, which correspond to different fields of view. In specific, the neural network in the shared-weights scaling sub-network 211 includes a convolutional layer 241 with a kernel size of 7×7, three residual blocks 242, and a channel attention layer 243. In addition, the neural network in the shared-weights scaling sub-network 212 does not include a convolutional layer, but includes a pooling block 251, three residual blocks 252 and a channel attention layer 253. The neural network in the shared-weights scaling sub-network 213 includes two pooling blocks 261, two residual blocks 262 and a channel attention layer 263. In other embodiments, the shared-weights scaling sub-networks 211 to 213 may also add batch normalization, dropout, dilated convolution, depthwise separable convolution, non-local neural network, or the like, and the present disclosure is not limited thereto.

Since the numbers of pooling blocks in the shared-weights scaling sub-networks 211-213 are 0, 1, and 2, respectively, the pooling blocks are utilized to reduce the resolution of the image, which means that the fields of view of the shared-weights scaling sub-networks 211-213 are different. In the following, the shared-weights scaling sub-network 211 is expressed as a network N_{L}, the shared-weight scaling sub-network 212 is expressed as a network N_{M}, and the shared-weights scaling sub-network 213 is expressed as a network Ns, in which L, M, and S indicate large, medium and small, respectively. The shared-weights scaling sub-network 211 is utilized to process relatively regional features in the image, while the shared-weights scaling sub-networks 212 and 213 are utilized to process relatively global features in the image. In addition, the three neural networks of the shared-weights scaling sub-network 211 respectively output three feature vectors that are expressed as

$${f}_{L}^{A} , {f}_{L}^{B} , and {f}_{L}^{C}$$

respectively corresponding to the images I^{A}, I^{B}, and I^{C}. The feature

$${f}_{L}^{A}$$

is also referred to as a gastric antrum scaling feature, the feature vector

$${f}_{L}^{B}$$

is also referred to as a gastric body scaling feature, and the feature vector

$${f}_{L}^{C}$$

is also referred to as a gastric cardia scaling feature. Similarly, the three neural networks of the shared-weights scaling sub-network 212 respectively output three feature vectors that are a gastric antrum scaling feature

$${f}_{M}^{A} ,$$

a gastric cardia scaling feature

$${f}_{M}^{B} ,$$

and a gastric cardia scaling feature

$${f}_{M}^{C} ,$$

respectively. The three neural networks of the shared-weights scaling sub-network 213 outputs three feature vectors, respectively, expressed as a gastric antrum scaling feature

$${f}_{S}^{A} ,$$

a gastric body scaling feature

$${f}_{S}^{B} ,$$

and a gastric cardia scaling feature

$${f}_{S}^{C} .$$

Next, the scaling feature fusion module 210 concatenates the gastric antrum scaling features

$${f}_{L}^{A} , {f}_{M}^{A} , and {f}_{S}^{A}$$

output by all shared-weights scaling sub-networks 211-213 to obtain a cross-view gastric antrum feature f^{A}, which is expressed as Formula 1 below. Such cross-view gastric antrum feature f^{A} includes the characteristics of the gastric antrum in different fields of view.

$${f}^{A} = \{ {f}_{L}^{A} , {f}_{M}^{A} , {f}_{S}^{A} \} \qquad [ Formula 1 ]$$

Similarly, the scaling feature fusion module 210 concatenates the gastric body scaling features

$${f}_{L}^{B} , {f}_{M}^{B} , and {f}_{S}^{B}$$

output by all shared-weights scaling sub-networks 211-213 to obtain a cross-view gastric body feature f^{B}, which is expressed as Formula 2 below.

$${f}^{B} = \{ {f}_{L}^{B} , {f}_{M}^{B} , {f}_{S}^{B} \} \qquad [ Formula 2 ]$$

Similarly, the scaling feature fusion module 210 concatenates the gastric cardia scaling features

$${f}_{L}^{C} , {f}_{M}^{C} , and {f}_{S}^{C}$$

output by all shared-weights scaling sub-networks 211-213 to obtain a cross-view gastric cardia feature f^{C}, which is expressed as Formula 3 below.

$${f}^{C} = \{ {f}_{L}^{C} , {f}_{M}^{C} , {f}_{S}^{C} \} \qquad [ Formula 3 ]$$

In the above embodiment, the gastric antrum endoscopic image, the gastric body endoscopic image, and the gastric cardia endoscopic image are utilized, but in other embodiments, endoscopic images of other sections may also be added. While an image is added, a corresponding neural network may also be added to each shared-weights scaling sub-network 211-213. The newly added neural network may share weights with other neural networks, and then the features corresponding to the same sections output by different shared-weights scaling sub-networks 211-213 may be concatenated together, as expressed by Formulas 1-3. In other words, the number of neural networks is the same as the number of input images and the number of cross-view features output by the scaling feature fusion module 210, and the number of the shared-weights scaling sub-networks 211-213 is the same as the number of fields of view. Those with ordinary knowledge in the art may slightly modify the network architecture shown in FIG. 2 based on the above content.

In the above method, the gastric antrum, the gastric body, and the gastric cardia may be considered simultaneously under different fields of view. Combining these cross-view features may simultaneously consider regional and global features, which avoids the problem of over-fitting and is more representative and discriminative.

Next, the cross-view gastric antrum feature f^{A}, the cross-view gastric antrum feature f^{B}, and the cross-view gastric cardia feature f^{C} are input to the section correlation module 220. The section correlation module 220 concatenates at least two of the features f^{A}, f^{B}, and f^{C} based on medical insights, which considers the definition of the corpus-predominant gastritis index. In general, the degree of inflammation in the gastric cardia is compared with the degree of inflammation in the antrum, and the degree of inflammation in the gastric body is compared with the degree of inflammation in the antrum. Accordingly, in the present disclosure, the cross-view gastric antrum feature f^{A} and the cross-view gastric body feature f^{B} are concatenated to form a feature f^{AB}, which is expressed as Formula 4 below.

$${f}^{AB} = \{ {f}^{A} , {f}^{B} \} \qquad [ Formula 4 ]$$

In addition, the cross-view gastric antrum feature f^{A} and the cross-view gastric cardia feature f^{C} are concatenated to form a feature f^{AC}, which is expressed as Formula 5 below.

$${f}^{A C} = \{ {f}^{A} , {f}^{C} \} \qquad [ Formula 5 ]$$

In addition, in order to consider all sections in the calculation of the corpus-predominant gastritis index, the cross-view gastric antrum feature f^{A}, the cross-view gastric body feature f^{B}, and the cross-view gastric cardia feature f^{C} are concatenated to form a feature f^{ABC}, which is expressed as Formula 6 below.

$${f}^{ABC} = \{ {f}^{A} , {f}^{B} , {f}^{C} \} \qquad [ Formula 6 ]$$

The section correlation module 220 also includes networks 271-273. Each of the networks 271-273 includes two 3×3 convolutional layers and two fully-connected layers. The above-mentioned feature f^{AB} is input to the network 271 to generate a first fusion feature; the above-mentioned feature f^{ABC} is input to the network 272 to generate a second fusion feature; the above-mentioned feature f^{AC} is input to the network 273 to generate a third fusion feature.

The first fused feature may be input to a classifier (not shown) to calculate a loss 281 (also expressed as [symbol] ^{AB}), which is expressed as Formula 7 below.

$${ℓ}^{AB} = {\sum }_{k = 1}^{K} - {y}_{k} {log}_{2} ( {p}_{k}^{AB} ) , \qquad [ Formula 7 ]$$

where K represents the number of all training data.

$${p}_{k}^{AB}$$

is the output of the classifier for the k^{th} training data. yₖ is the ground truth of the k^{th} training data, please refer to the thesis “Tsai, Y-C., et al. “The corpus-predominant gastritis index may serve as an early marker of Helicobacter pylori-infected patients at risk of gastric cancer.” Alimentary pharmacology & therapeutics 37.10 (2013): 969-978.”

Similarly, the above-mentioned second fusion feature is input to another classifier to calculate a loss 282 (also expressed as [symbol] ^{ABC}), which is expressed as Formula 8 below, where

$${p}_{k}^{ABC}$$

is the output of this classifier.

$${ℓ}^{ABC} = {\sum }_{k = 1}^{K} - {y}_{k} {log}_{2} ( {p}_{k}^{ABC} ) \qquad [ Formula 8 ]$$

The above-mentioned third fusion feature is input to another classifier to calculate a loss 283 (also expressed as [symbol] ^{AC}), which is expressed as Formula 9 below, where

$${p}_{k}^{A C}$$

is the output of this classifier.

$${ℓ}^{A C} = {\sum }_{k = 1}^{K} - {y}_{k} {log}_{2} ( {p}_{k}^{A C} ) \qquad [ Formula 9 ]$$

In addition, the above-mentioned first, second, and third fusion features are concatenated to obtain a sum feature. The sum feature is input to a classifier 274 to calculate a loss 284 (also expressed as [symbol] ^{F}), which is expressed as Formula 10 below.

$${ℓ}^{F} = {\sum }_{k = 1}^{K} - {y}_{k} {log}_{2} ( {p}_{k}^{F} ) , \qquad [ Formula 10 ]$$

where

$${p}_{k}^{F}$$

is the output of the classifier 274, which is also the corpus-predominant gastritis index. In other words, the classifier 274 calculates the corpus-predominant gastritis index based on the above sum feature. In the training stage, the above losses are considered at the same time, and a sum loss [symbol] is expressed as Formula 11 below.

$$ℓ = {ℓ}^{AB} + {ℓ}^{A C} + {ℓ}^{ABC} + {ℓ}^{F} \qquad [ Formula 11 ]$$

In the process of minimizing the sum loss [symbol] , the above-mentioned scaling features representing different fields of view and the above-mentioned fusion features combining different sections may be integrated to represent the inflammation status more effectively to calculate the corpus-predominant gastritis index.

In other embodiments, the networks 271-273 and the classifier 274 may also adopt other architectures, such as architectures including residual blocks, batch normalization, or the like, and the present disclosure is not limited to the embodiment of FIG. 2.

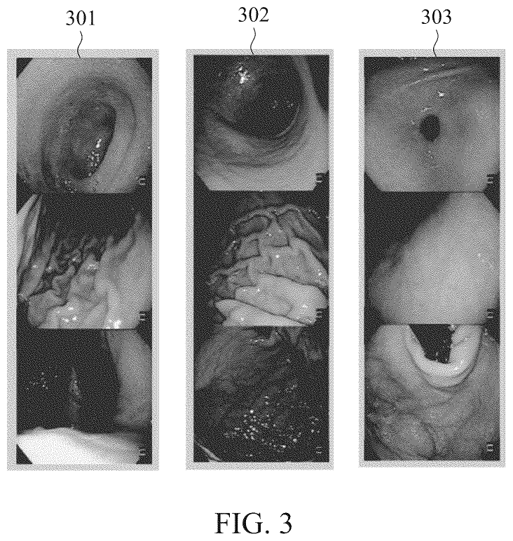

FIG. 3 is a schematic diagram of detection results in accordance with some embodiments of the present disclosure. Referring to FIG. 3, three testing data 301-303 are illustrated in FIG. 3. Each of the testing data includes a gastric antrum endoscopic image, a gastric body endoscopic image, and a gastric cardia endoscopic image, and patients with the testing data 301-303 have been diagnosed with gastric body inflammation. Based on the method proposed above, the calculated probabilities of gastric body inflammation in the three testing data are 0.9999, 0.9903, and 0.9999, respectively, which is consistent with the real output.

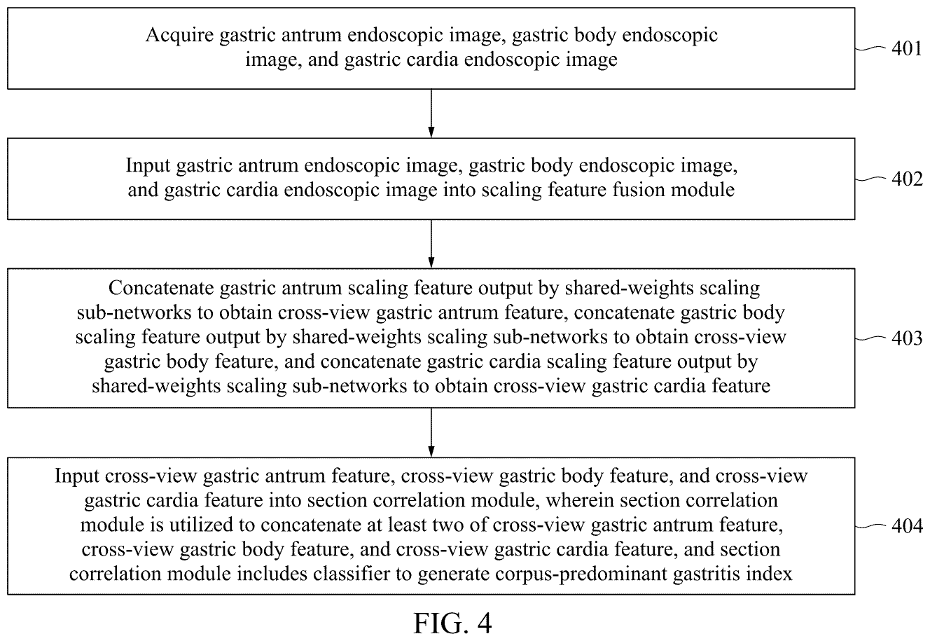

FIG. 4 is a flowchart of a method for analyzing a gastric endoscopic image in accordance with some embodiments of the present disclosure. Referring to FIG. 4, which includes Steps 401-404. In Step 401, a gastric antrum endoscopic image, a gastric body endoscopic image, and a gastric cardia endoscopic image are acquired. In Step 402, the gastric antrum endoscopic image, the gastric body endoscopic image, and the gastric cardia endoscopic image are input to a scaling feature fusion module. The scaling feature fusion module contains a plurality of shared-weights scaling sub-networks. The fields of view of these shared-weights scaling sub-networks are different from each other. Each of the shared-weights scaling sub-networks is utilized to receive the gastric antrum endoscopic image, the gastric body endoscopic image, and the gastric cardia endoscopic image, and utilized to output a gastric antrum scaling feature, a gastric body scaling feature and a gastric cardia scaling feature. In Step 403, the gastric antrum scaling features output by the shared-weights scaling sub-network are concatenated to obtain a cross-view gastric antrum feature, the gastric body scaling features output by the shared-weights scaling sub-network are concatenated to obtain a cross-viewing gastric body feature, and the gastric cardia scaling features output by the shared-weights scaling sub-network are concatenated to obtain a cross-viewing gastric cardia feature. In Step 404, the cross-view gastric antrum feature, the cross-view gastric body feature, and the cross-view gastric cardia feature are input to a section correlation module. The section correlation module is utilized to concatenate at least two of the cross-view gastric antrum feature, the cross-view gastric body feature, and the cross-view gastric cardia feature, and the section correlation module includes a classifier to generate a corpus-predominant gastritis index. Each step in FIG. 4 has been described in detail above and is not repeated herein. It should be noted that each step in FIG. 4 may be implemented as multiple program codes or circuits, and the present disclosure is not limited thereto. In addition, the method in FIG. 4 may be utilized in conjunction with the above embodiments or may be utilized alone. In other words, other steps may also be added between the steps in FIG. 4.

It will be apparent to those skilled in the art that various modifications and variations can be made to the structure of the present disclosure without departing from the scope or spirit of this disclosure. In view of the foregoing, it is intended that the present disclosure covers modifications and variations of this disclosure provided they fall within the scope of the following claims.

What is claimed is:

1. A method for analyzing a gastric endoscopic image, comprising:
acquiring a gastric antrum endoscopic image, a gastric body endoscopic image, and a gastric cardia endoscopic image;
inputting the gastric antrum endoscopic image, the gastric body endoscopic image, and the gastric cardia endoscopic image into a scaling feature fusion module which contains a plurality of shared-weights scaling sub-networks, each of the shared-weights scaling sub-networks having a different field of view, and utilized to receive the gastric antrum endoscopic image, the gastric body endoscopic image, and the gastric cardia endoscopic image, and output a gastric antrum scaling feature, a gastric body scaling feature, and a gastric cardia scaling feature, respectively;
concatenating the gastric antrum scaling feature output by the shared-weights scaling sub-networks to obtain a cross-view gastric antrum feature, concatenating the gastric body scaling feature output by the shared-weights scaling sub-networks to obtain a cross-view gastric body feature, and concatenating the gastric cardia scaling feature output by the shared-weights scaling sub-networks to obtain a cross-view gastric cardia feature; and
inputting the cross-view gastric antrum feature, the cross-view gastric body feature, and the cross-view gastric cardia feature into a section correlation module, wherein the section correlation module is utilized to concatenate at least two of the cross-view gastric antrum feature, the cross-view gastric body feature, and the cross-view gastric cardia feature, and the section correlation module comprises a classifier to generate a corpus-predominant gastritis index (CGI).

2. The method of claim 1, wherein one of the shared-weights scaling sub-networks comprises a plurality of neural networks, the neural networks share weights and are utilized to receive the gastric antrum endoscopic image, the gastric body endoscopic image, and the gastric cardia endoscopic image, respectively.

3. The method of claim 2, wherein one of the neural networks comprises a convolutional layer, a residual block, a channel attention layer, or a pooling block.

4. The method of claim 1, wherein the section correlation module is utilized to concatenate the cross-view gastric antrum feature and the cross-view gastric body feature to generate a first fusion feature,
wherein the section correlation module is utilized to concatenate the cross-view gastric antrum feature, the cross-view gastric body feature, and the cross-view gastric cardia feature to generate a second fusion feature, and
wherein the section correlation module is utilized to concatenate the cross-view gastric antrum feature and the cross-view gastric cardia feature to generate a third fusion feature.

5. The method of claim 4, wherein the first fusion feature is input to the classifier to calculate a first loss, and wherein the second fusion feature is input to the classifier to calculate a second loss, and wherein the third fusion feature is input to the classifier to calculate a third loss.

6. The method of claim 5, wherein the section correlation module is utilized to concatenate the first fusion feature, the second fusion feature, and the third fusion feature to obtain a sum feature,
wherein the classifier is utilized to generate the corpus-predominant gastritis index based on the sum feature.

7. The method of claim 6, wherein the sum feature is input to the classifier to calculate a fourth loss.

8. The method of claim 7, wherein the first loss, the second loss, the third loss and the fourth loss are summed into a sum loss.

9. A computer system, comprising:
a memory configured to store a plurality of instructions; and
a processor coupled to the memory and configured to execute the instructions to perform the following steps:
acquiring a gastric antrum endoscopic image, a gastric body endoscopic image, and a gastric cardia endoscopic image;
inputting the gastric antrum endoscopic image, the gastric body endoscopic image, and the gastric cardia endoscopic image into a scaling feature fusion module which contains a plurality of shared-weights scaling sub-networks, each of the shared-weights scaling sub-networks having a different field of view, and utilized to receive the gastric antrum endoscopic image, the gastric body endoscopic image, and the gastric cardia endoscopic image, and output a gastric antrum scaling feature, gastric body scaling feature, and a gastric cardia scaling feature, respectively;
concatenating the gastric antrum scaling feature output by the shared-weights scaling sub-networks to obtain a cross-view gastric antrum feature, and concatenating the gastric body scaling feature output by the shared-weights scaling sub-networks to obtain a cross-view gastric body feature, and concatenating the gastric cardia scaling feature output by the shared-weights scaling sub-networks to obtain a cross-view gastric cardia feature; and
inputting the cross-view gastric antrum feature, the cross-view gastric body feature, and the cross-view gastric cardia feature into a section correlation module, wherein the section correlation module is utilized to concatenate at least two of the cross-view gastric antrum feature, the cross-view gastric body feature, and the cross-view gastric cardia feature, and the section correlation module comprises a classifier to generate a corpus-predominant gastritis index (CGI).

10. The computer system of claim 9, wherein one of the shared-weights scaling sub-networks comprises a plurality of neural networks, the neural networks share weights and utilized to receive the gastric antrum endoscopic image, the gastric body endoscopic image, and the gastric cardia endoscopic image, respectively.

11. The computer system of claim 10, wherein one of the neural networks comprises a convolutional layer, a residual block, a channel attention layer, or a pooling block.

12. The computer system of claim 9, wherein the section correlation module is utilized to concatenate the cross-view gastric antrum feature and the cross-view gastric body feature to generate a first fusion feature,
wherein the section correlation module is utilized to concatenate the cross-view gastric antrum feature, the cross-view gastric body feature, and the cross-view gastric cardia feature to generate a second fusion feature, and
wherein the section correlation module is utilized to concatenate the cross-view gastric antrum feature and the cross-view gastric cardia feature to generate a third fusion feature.

13. The computer system of claim 12, wherein the first fusion feature is input to the classifier to calculate a first loss, and wherein the second fusion feature is input to the classifier to calculate a second loss, and wherein the third fusion feature is input to the classifier to calculate a third loss.

14. The computer system of claim 13, wherein the section correlation module is utilized to concatenate the first fusion feature, the second fusion feature, and the third fusion feature to obtain a sum feature,
wherein the classifier is utilized to generate the corpus-predominant gastritis index based on the sum feature.

15. The computer system of claim 14, wherein the sum feature is input to the classifier to calculate a fourth loss.

16. The computer system of claim 15, wherein the first loss, the second loss, the third loss and the fourth loss are summed into a sum loss.
